(19) United States
(12) Patent Application Publication
(10) Pub. No.: US 2012/0232580 A1
(43) Pub. Date: Sep. 13, 2012

(54) SURGICAL FORCEPS HAVING ENGAGEMENT IN A GROOVE

(75) Inventor: Thomas Aue, Wedel (DE)

(73) Assignee: OLYMPUS WINTER & IBE GMBH, Hamburg (DE)

(21) Appl. No.: 13/500,498

(22) PCT Filed: Nov. 9, 2010
(86) PCT No.: PCT/EP2010/006803

§ 371 Date: May 23, 2012

(30) Foreign Application Priority Data
Nov. 26, 2009 (DE) 10 2009 055 747.4

(51) Int. Cl. A61B 17/28

(52) U.S. Cl. 606/206

(57) ABSTRACT

Surgical forceps are provided having an elongated shaft which at its distal end bears a forceps jaw having two jaw parts which are movable relative to one another, and at its proximal end bears an actuating handle which by means of a longitudinally displaceable actuating rod that passes through the shaft controls the relative motion of the jaw parts via the engagement of at least one cam on the forceps jaw or on the actuating rod in a groove which is inclined at an angle with respect to the direction of displacement of the actuating rod on the shaft or on the forceps jaw. The groove is provided, at least in one section, with an angle of inclination which is smaller than the angle of inclination up to which self-locking in the groove occurs.

### BACKGROUND

I. Field of the Invention

The invention relates to forceps.

II. Description of the Related Art

DE 195 21 257 A1 discloses generic forceps having a groove in engagement with a cam and situated at a fairly large angle of inclination with respect to the direction of displacement of the actuating rod. The groove engagement may transmit motions in both directions. Due to the large angle of the groove with respect to the direction of displacement, there is no self-locking. Large changes in the angle of the moved jaw part may be quickly made on account of the large angle.

A disadvantage of this known design, however, is that for closing forces to be applied by the forceps jaw, for example when a needle holder is used, the clamping force for holding the needle must be continuously maintained by applying an actuating force via the actuating rod. For this purpose, in the prior art it is known to provide a fixing device in the actuating handle.

### SUMMARY

Exemplary embodiments of the invention provide generic forceps which are easier to operate.

According to embodiments of the invention, the angle of inclination of the groove, at least in a partial region, is less than the angle up to which self-locking occurs. When the cam is located in this partial region of the groove, self-locking is present. This means that when the actuating rod is actuated, a motion in the groove is possible, but not in the reverse path when the jaw part is moved. Thus, the moved jaw part may be acted on by the actuating rod, but not in the reverse direction, since self-locking is present for this direction. The self-locking ensures that, after an object is clamped in the forceps jaw by actuating the actuating rod, the clamped position is maintained even when the actuating rod is released. The self-locking thus results in locking of the forceps jaw in the clamped state. This means that for a needle holder, for example, the needle may be gripped and clamped with sufficient closing force. The actuating handle may then be released while maintaining the clamped position in which the needle is securely held. The angle of inclination up to which self-locking occurs is a few degrees, depending on the coefficients of friction of the materials used.

The groove may have a self-locking design over its entire length. However, it is advantageous for only a first end region to have a self-locking design, while another end region with a larger angle of inclination does not have a self-locking design. The forceps jaw may thus be moved quickly over fairly large angular ranges in the second end region with a smaller transmission ratio. When the forceps jaw is closed, the cam passes into the self-locking first end region, so that self-locking occurs at that location and the forceps may be released in the clamped state. The kinematics of the forceps are to be selected in such a way that for a clamped object, a held needle, for example, the cam actually passes into the self-locking first end region.

A spring for cushioning the actuating force is advantageous for surgical forceps when these are to be locked in the gripping position. The spring is able to maintain the closing force, even when the gripped object, for example tissue, continuously gives way, for example, undergoes shrinkage during electrocoagulation. In addition, such a spring is able to maintain the force when a rigid body is gripped, for example when the forceps are used as a needle holder. In known surgical forceps, the actuating force is continuously transmitted through the actuating rod. The spring may therefore be situated in the handle. For the forceps according to embodiments of the invention, it is advantageous to provide such a spring in the forceps jaw, at which location the spring is able to maintain the actuating force when the forceps jaw itself, as the result of engagement of the cam, is locked at the first end region of the groove. For this purpose, in one simple example one of the jaw parts has an elastically resilient design.

According to embodiments of the invention, it is advantageous that the forceps jaw is rotatably supported with respect to the actuating handle, thus enhancing the possible uses of the forceps.

Known surgical forceps having a rotatable forceps jaw have the disadvantage that the actuating force is transmitted from the actuating handle to the distally situated forceps jaw, and therefore the counterforce transmitted via the shaft passes through a rotary bearing, which is blocked by this force. Thus, according to the prior art, rotating the forceps jaw while transmitting the actuating force at the same time can be achieved only by rotating the complete forceps. In the forceps according to the invention, the shaft is free of axial forces, even when a closing force is applied between the jaw parts, so that the jaw is able to easily rotate. According to embodiments of the invention, it is advantageous to provide the rotary bearing at the distal end of the shaft. Co-rotation of the shaft while the forceps jaw is rotating is thus avoided. Rotational friction of the shaft, for example in a trocar seal, is avoided in this way. In addition, curved shafts, which usually are not rotatable as a whole at the installation site, may be used.

According to embodiments of the invention, particularly advantageous rotational control from the actuating handle is achieved via the actuating rod, thus simplifying the design.

According to embodiments of the invention, the shaft of the forceps is advantageously curved. Such a curved shaft simplifies handling of the forceps during minimally invasive surgical procedures.

### BRIEF DESCRIPTION OF THE DRAWINGS

Embodiments of the invention are schematically illustrated by way of example in the drawings, which show the following:

FIG. 1 shows a side view of forceps according to the invention; and

FIG. 2 shows a partially cut away, enlarged illustration of the distal end region of the forceps in FIG. 1.

### DETAILED DESCRIPTION OF EXEMPLARY EMBODIMENTS

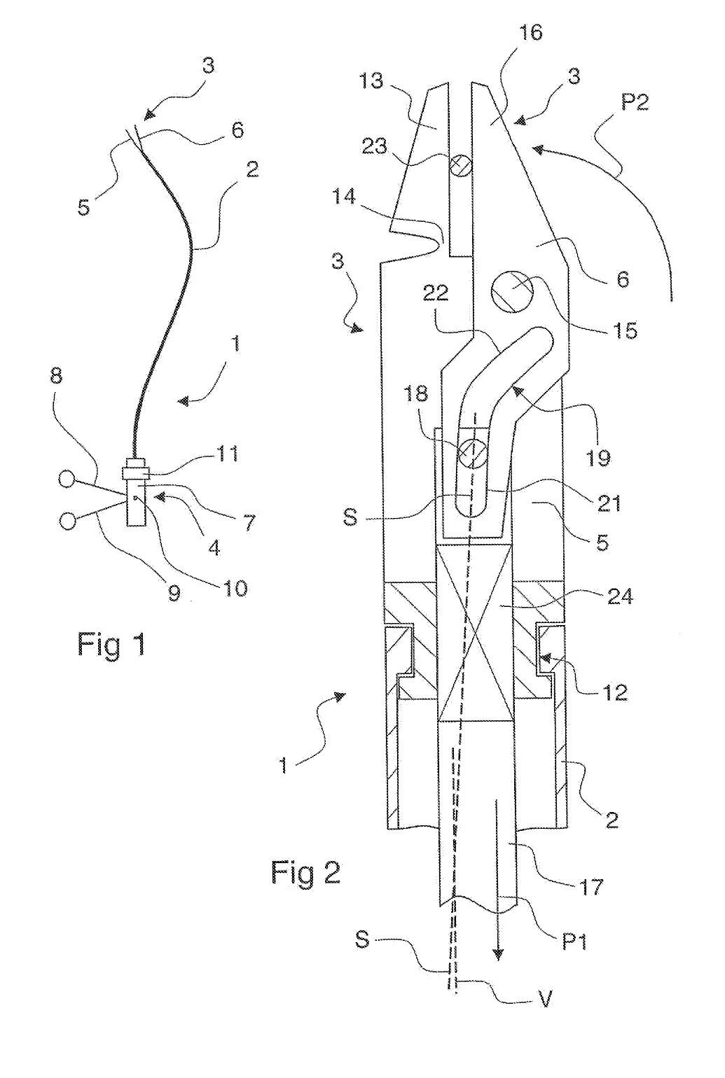

The forceps 1 illustrated in a side view in FIG. 1 are designed in particular for laparoscopic use. The forceps have an elongated, curved shaft 2 having a forceps jaw 3 at its distal end and an actuating handle 4 at its proximal end.

The forceps jaw 3 has two jaw parts, one jaw part 5 being stationarily attached, and the other jaw part 6 being pivotably attached, to the shaft 2.

The actuating handle 4 has a main body 7 on which two finger grips 8, 9, each having a holding ring as illustrated, are supported about an axis 10. In addition, a rotating ring 11 is supported on the main body 7.

The distal end region of the forceps 1 in FIG. 1 is illustrated in FIG. 2 in greatly enlarged form.

The shaft 2 has the cross-sectional design of a tube. As shown in the sectional illustration, the shaft is rotatably supported, via a rotary bearing 12, on the proximal end region of the stationary jaw part 5. The stationary jaw part 5 extends from the rotary bearing 12 to its distal end, where it has the design of a gripping jaw 13 which is connected to the remaining portion of the jaw part 5 via a weakened, elastic location 14. Also attached to the jaw part 5 is an axial pin 15 on which the pivotable jaw part 6, which in its distal end region is likewise designed as a gripping jaw 16, is pivotably supported.

An actuating rod 17 is supported within the shaft 2 so as to be longitudinally displaceable, and at its proximal end (not illustrated) is connected in a longitudinally adjustable manner in the main body 7 of the actuating handle 4, to be displaced when the finger grips 8, 9 are actuated.

The actuating rod 17 is supported by a polygonal region 24 in a rotationally fixed manner, but so as to be longitudinally displaceable in the proximal end region of the stationary jaw part 5. In addition, at the actuating handle 4, the actuating rod 17 has a longitudinally displaceable rotary coupling with the rotating ring 11 in a manner not illustrated. Thus, when the rotating ring 11 rotates, the stationary jaw part 5 in the rotary bearing 12 is brought into co-rotation.

A laterally projecting cam 18 which extends in a groove 19 provided in the pivotable jaw part 6 is mounted at the distal end of the actuating rod 17. The groove 19 has a first end region 21 and a second end region 22.

The first end region 21 extends in the direction of the dashed slanted line S which, as shown in FIG. 2, defines a very small oblique angle, the angle of inclination, with respect to the direction of displacement V of the actuating rod 17. On the other hand, the second end region 22 of the groove 19, as shown in FIG. 2, has a very large angle of inclination.

When the cam 18 is moved in the second end region 22, very large pivoting motions of the pivotable jaw part 6 result from a small displacement of the actuating rod 17. In contrast, only very small pivoting motions result when the cam 18 is moved in the first end region 21 of the groove 19.

The kinematics of the forceps illustrated in FIG. 2 are such that when the actuating rod 17 is retracted in the direction of the arrow P1, the gripping jaw 16 is pivoted toward the stationary gripping jaw 13 in the direction of the arrow P2. When the cam 18 moves within the second end region 22, initially there is rapid pivoting with low force. In the cause of this, initially there is rapid pivoting with low force when the cam 18 moves within the second end region 22. As the actuating rod 17 is retracted further, the cam 18 passes into the first end region 21 and then brings about very small pivoting motions, but with high force transmission.

As illustrated in FIG. 2, the forceps 1 according to embodiments of the invention are particularly suited as a needle holder for enabling a needle 23 to be held between the gripping jaws 13 and 16 with great force.

The forceps 1 have a design of the gripping jaws 13 and 16 so that when the needle 23 is held in the manner illustrated in FIG. 2, the cam 18 is able to pass into the first end region 21, as illustrated in FIG. 2. The needle 23 may then be held between the gripping jaws 13 and 16 with great force, with only a very low force on the actuating rod 17 being necessary.

The angle of inclination of the groove 19 in the first end region 21 may also be zero. The actuating rod 17 may then be entirely free of force when the gripping jaws 13 and 16 are holding with great force. When the actuating rod 17 is free of force, i.e., acted on by only a low closing force, the rotary bearing 12 is free of axial load, which would hinder its free rotatability. The forceps jaw 3 may therefore be rotated very easily, even under high gripping forces, which is very useful when a securely held needle 23 is to be pivoted into a specific suturing position.

The angle of inclination between the dashed lines S and V, i.e., between the direction of the first end region 21 of the groove 19 and the direction of displacement of the actuating rod 17, is very small, as illustrated in FIG. 2. The angle of inclination is selected in such a way that it causes self-locking, so that when the cam 18 is pulled into the first end region 21 by retraction of the actuating rod 17, the cam 18 is clamped at that location, and prevents independent opening of the forceps jaw 3 even when the actuating rod 17 is under no load at all.

For the forceps 1 according to an embodiment of the invention as illustrated in FIG. 2, it is possible to maintain high closing forces in the forceps jaw 3 without these forces having to be transmitted from the actuating handle 4 through the shaft 2. The forceps jaw 3 is therefore blocked in situ, i.e., by itself.

Cushioning of the actuating force by means of a spring is helpful for many purposes, for example to ensure the closing force for a very rigid object such as the needle 23, or to maintain this closing force when the held object diminishes. For this purpose, a spring is usually provided in the actuating handle 4.

In the design according to an embodiment of the invention in FIG. 2, in which the forceps jaw 3 is blocked by self-locking by itself, such a spring must be provided in the jaw part. In the illustrated exemplary embodiment, for this purpose one of the two jaw parts, namely, the stationary jaw part 5, is provided with the spring 14, which in the exemplary embodiment is designed as a weakened point between the jaw part 5 and its gripping jaw 13. The spring may also be designed simply as elastic resilience of one of the jaw parts.

In the illustrated exemplary embodiment, the jaw part 5 has a stationary design, and the jaw part 6 has a design which is pivotable about the axial pin 15. However, a design having two pivotable jaw parts may also be used.

1. Surgical forceps, comprising:
an elongated shaft;
a forceps jaw disposed at a distal end of the elongated shaft, the forceps jaw having two jaw parts which are movable relative to one another;
an actuating handle disposed at a proximal end of the elongated shaft; and
a longitudinally displaceable actuating rod that passes through the elongated shaft; wherein
the actuating handle, by means of the actuating rod, controls a relative motion of the jaw parts via engagement of at least one cam on the forceps jaw or on the actuating rod respectively, in a groove on the actuating rod or on the forceps jaw respectively,
the groove is inclined at an angle with respect to a direction of displacement of the actuating rod, and
the groove is provided, in at least one section, with an angle of inclination which is smaller than an angle of inclination up to which self-locking in the groove occurs.

2. The surgical forceps according to claim 1, wherein:
in a first end region, the groove has a self-locking design, and
in a second end region, the groove has a larger angle of inclination and does not have the self-locking design.

3. The surgical forceps according to claim 1, wherein a spring which yields when an actuating force is applied, is situated in the forceps jaw.

4. The surgical forceps according to claim 1, wherein the forceps jaw is rotatable with respect to the actuating handle.

5. The surgical forceps according to claim 4, wherein the forceps jaw is rotatably supported on the distal end of the elongated shaft.

6. The surgical forceps according to claim 4, wherein the actuating rod is rotationally controllable from the actuating handle and is in rotationally fixed engagement with the forceps jaw.

7. The surgical forceps according to claim 1, wherein the elongated shaft has a curved design.
